(19) United States
(12) Patent Application Publication
(10) Pub. No.: US 2005/0187181 A1
(43) Pub. Date: Aug. 25, 2005

(54) Use of purine nucleosides to stimulate Na/K ATPase and to treat or prevent shock

(75) Inventors: Donald S. Gann, Lutherville, MD (US); Daniel N. Darlington, Catonsville, MD (US)

Correspondence Address: EVANS & MOLINELLI, PLLC, 6411 CHAPEL VIEW ROAD, CLIFTON, VA, 20124, US

(73) Assignee: University of Maryland, Baltimore, MD (US)

(21) Appl. No.: 11/061,938

(22) Filed: Feb. 19, 2005

Related U.S. Application Data
(60) Provisional application No. 60/598,396, filed on Aug. 3, 2004.
(60) Provisional application No. 60/546,411, filed on Feb. 20, 2004.

(51) Int. Cl. A61K 31/7076

(52) U.S. Cl. 514/45

(57) ABSTRACT

This invention relates to methods of treating or preventing hemorrhagic and septic shock in an animal by administering inosine, guanosine, deoxyinosine, deoxyguanosine or a mixture thereof. Other purine nucleosides or analogs are described that have therapeutic use in treating or preventing shock. The invention also describes methods for increasing Na/K ATPase activity in erythrocytes or other cells in an animal having below normal activity of this enzyme by administering inosine, guanosine, deoxyinosine, deoxyguanosine or a mixture thereof.

### CROSS-REFERENCE TO RELATED APPLICATIONS

This application claims benefit of Provisional Application 60/598,396, filed Aug. 3, 2004 and on 60/546,411 filed on Feb. 20, 2004, the entire contents of which are hereby incorporated by reference as if fully set forth herein, under 35 U.S.C. § 119(e).

### STATEMENT OF GOVERNMENTAL INTEREST

This invention was made with Government support NIH grant RO1—HL57490 and the R Adams Cowley Shock Trauma Center. The Government has certain rights in the invention.

### BACKGROUND OF THE INVENTION

1. Field of the Invention

The present invention relates to the therapeutic use of intravenously administered purine nucleosides, especially inosine, guanosine, deoxyinosine, deoxyadenosine and deoxyguanosine to prevent and treat hemorrhagic shock and septic shock.

2. Description of the Related Art

Severe hemorrhagic shock is associated with an elevation in plasma potassium, a decrease in transmembrane potential and an increase in cell swelling (1-13), all of which have been attributed to decreased activity of sodium-potassium adenosine triphosphatase (Na/K ATPase) by a circulating inhibitor whose effect appears during shock (10-14). Shock caused by sepsis or cardiac insufficiency is also associated with a decrease in the activity of Na/K ATPase. Na/K ATPase is defined operationally as that fraction of Na and K transport that can be inhibited by ouabain. Endogenous inhibitors of Na/K ATPase have been reported by many laboratories (20,27-30) including ours (11-13, 21). These endogenous inhibitors appear in plasma, urine and tissue in various clinical conditions including hemorrhagic and septic shock, heart failure, myocardial depression and diabetes (11-13, 27, 28). Shires et al (9) reported that septic shock in adult baboons induced a decrease in membrane potential in skeletal muscle and in erythrocytes (RBCs) along with a rise in intracellular sodium (Na), chloride (Cl) and water, and decrease in intracellular K. Our laboratory has also reported depolarization and cell swelling in both hemorrhagic and septic shock in rats and dogs (10-13, 21). The movement of water, Na and Cl into cells and out of the extracellular space contributes to the fall in blood volume and blood pressure seen in hemorrhagic shock, and probably explains why resuscitation with volumes larger than those lost during hemorrhage are necessary for cardiovascular stabilization.

Hemorrhagic shock is a life-threatening condition brought on by severe blood loss. For example, hemorrhagic shock may originate from internal or external hemorrhage, gun shot wounds, severe trauma or any other condition associated with blood loss. Unfortunately, because of the severity and complexity of hemorrhagic shock, a patiert is likely to die unless treated during a relatively short treatment window, generally known as the “golden hour”.

Hemorrhagic shock is an extremely complex process, stimulating multiple injury pathways. That is, the pathophysiology of hemorrhagic shock is multifactorial. For example, hemorrhagic shock stimulates the release of cytokines and nitric oxide, as well as the formation of peroxynitrite and the generation of superoxide radical. In addition, hemorrhagic shock stimulates pathways that release platelet activating factor, and induces alterations in the complement cascade and coagulation cascade. Furthermore, hemorrhagic shock stimulates neuroendocrine responses, electrolyte disturbances and metabolic changes. Accordingly, the treatment and management of a patient experiencing hemorrhagic shock is extremely complex.

The initial phase of hemorrhagic shock, unless rapidly corrected, is followed by progressive tissue ischemia, end-organ dysfunction and refractory vascular failure. Hemorrhagic shock also is associated with early vasomotor paralysis and cardiovascular collapse. Accordingly, conventional resuscitation methods have been directed toward hemostasis and intravenous infusion of sufficiently large volumes of fluid, preferably blood, in order to restore cardiac index, improve oxygen-carrying capacity and minimize cellular hypoxia.

Infection remains a major cause of morbidity and mortality after severe hemorrhagic episodes. Following hemorrhagic shock, the translocation of enteric bacteria to extraintestinal sites frequently contributes to sepsis and increases the risk of severe illness. Prolonged susceptibility to infection is often seen in subjects who have initially recovered from hemorrhagic shock. Hemorrhagic shock also may cause suppression or reduction of various immune functions, thus rendering the subject less able to cope with an increased bacterial influx and further endangering the subject. Therapy, such as intravenous antibiotic therapy and massive fluid infusion, which is directed towards alleviating post-shock infection, is of limited effectiveness because the initial damage, translocation of enteric bacteria and suppression of the immune response, have already been sustained.

Septic shock (also known as sepsis) causes more than 150,000 deaths annually in the United States. Sepsis is defined as a clinical disorder whose symptoms may include well defined abnormalities in body temperature, heart rate, breathing rate, white blood cell count, hypotension, organ perfusion abnormalities, and multiple organ dysfunction. It may be caused by bacterial (either gram negative or gram positive), fungal, viral and other infections as well as by non-infective stimuli such as multiple trauma, severe burns, organ transplantation and pancreatitis. Even with improved patient management the mortality rate ranges from 50% to 75% in patients with established septic shock. There has not been a significant decrease in this mortality rate since the advent of broad spectrum antibiotics in the early 1960s. Septic patients usually die as a result of poor tissue perfusion and injury followed by multiple organ failure. It is now generally accepted that a significant portion of the peripheral responses occurring during septic shock are initiated by endotoxin. Endotoxin (also referred to herein as lipopolysaccharide, bacterial lipopolysaccharide or LPS), an outer membrane component of gram-negative bacteria, is released upon the death or multiplication of the bacteria. Although septic shock can follow any bacterial infection, it is most often the sequel to a gram negative infection. Klebsiella, Pseudomonas, Escherichia coli, Bacteroides and Salmonella are the most frequent cause.

Administration of endotoxin to experimental animals elicits a series of sequential cardiovascular, metabolic, and pathologic responses culminating in organ dysfunction and failure, ultimately resulting in death. When endotoxin is administered to normal human subjects, physiologic, biochemical, and cellular responses are induced that quantitatively mimic those occurring during septic shock. However, it is becoming increasingly recognized that the majority of responses observed during sepsis and endotoxemia are not due to direct actions of endotoxin, but result from endotoxin induction of a myriad of cellular and humoral inflammatory mediators. Furthermore, even with the vast research and clinical literature regarding sepsis and endotoxemia, there is no definitive regimen for the treatment of septic shock with the thrust of therapy being targeted at correction of symptoms. The impact of sepsis and any situation of endotoxemia is particularly devastating to patients with compromised cardiac and hepatic function and to immune compromised patients. Patients at high risk are the elderly (an increasing percentage of our society), chemotherapy patients, and those requiring surgery or invasive instrumentation. The current therapy of antibiotics and hemodynamic support has not proven to be successful. There is such an explosion of physiological responses and release of mediators during septic shock that the antagonism of a single mediator may not always be effective.

Septic shock usually begins with tremor, fever, falling blood pressure, rapid breathing and heart beat, and skin lesions. Within hours or days it can progress to spontaneous clotting in the blood vessels, severe hypotension, multiple organ failure and death. The component responsible for the toxic effect of the LPS molecule is the lipid component, called lipid A. This region is buried in the outer membrane of the bacterium and is believed to be reasonably constant between different species of gram negative bacteria. The polysaccharide region of the molecule extends from the surface of the bacterium and is different for each bacterial strain. The polysaccharide region consists of an inner core region composed of a heptose, and a 3, deoxy-D-manno-2-octulosonic acid (KDO) molecule. The KDO molecule is found in all lipopolysaccharide and links the polysaccharide to the lipid A moiety. The manner in which endotoxin evokes its effects is by binding to cells such as monocytes/macrophages or endothelial cells, and triggering them to produce various mediator molecules such as toxic oxygen radicals, hydrogen peroxide, tumor necrosis factor-alpha (TNF-.alpha.), various interleukins (IL-1, IL-6, IL-8 and IL-12). Endotoxin in even the very smallest amounts can activate these cells.

Due to the high fatality rate for hemorrhagic and septic shock there is a great need for new methods of preventing or treating these shock.

### REFERENCES

1. Darlington D N, Chew G, Ha T, et al. Corticosterone, but not glucose, treatment enables fasted adrenalectomized rats to survive moderate hemorrhage. Endocrinology 1990; 127:766-72.
2. Campion D S, Lynch L J, Rector F C, Jr., et al. G T. Effect of hemorrhagic shock on transmembrane potential. Surgery 1969; 66:1051-9.
3. Cunningham J N, Jr., Wagner Y, Shires G T. Changes in intracellular sodium content of red blood cells in hemorrhagic shock. Surg Forum 1970; 21:3840.
4. Shires G T, Cunningham J N, Backer C R, et al. Alterations in cellular membrane function during hemorrhagic shock in primates. Ann Surg 1972; 176:288-95.
5. Illner H P, Shires G T. Membrane defect and energy status of rabbit skeletal muscle cells in sepsis and septic shock. Arch Surg 1981; 116:1302-5.
6. Illner H, Shires G T. Changes in sodium, potassium, and adenosine triphosphate contents of red blood cells in sepsis and septic shock. Circ Shock 1982; 9:259-67.
7. Gallagher J F, Shires G T. Correlation of transmembrane potential difference with high-energy phosphate levels in hemorrhagic shock. Surg Forum 1977; 28:19-21.
8. Peitzman A B, Shires G T, 3rd, Illner H, et al. Effect of intravenous ATP-MgCl2 on cellular function in liver and muscular in hemorrhagic shock. Curr Surg 1981; 38:3004.
9. Shires G T, 3rd, Peitzman A B, Illner H, Shires G T. Changes in red blood cell transmembrane potential, electrolytes, and energy content in septic shock. J Trauma 1983; 23:769-74.
10. Boulanger B R, Evans J A, Lilly M P, et al. A circulating protein that depolarizes cells increases after hemorrhage in dogs. J Trauma 1993; 34:591-8; discussion 599.
11. Borchelt B D, Wright P A, Evans J A, et al. Cell swelling and depolarization in hemorrhagic shock. J Trauma 1995; 39:187-92; discussion 1924.
12. Eastridge B J, Darlington D N, Evans J A, et al. A circulating shock protein depolarizes cells in hemorrhage and sepsis. Ann Surg 1994; 219:298-305.
13. Evans J A, Darlington D N, Gann D S. A circulating factor(s) mediates cell depolarization in hemorrhagic shock. Ann Surg 1991; 213:549-56; discussion 556-7.
14. Kowluru R, Bitensky M W, Kowluru A, et al. Reversible sodium pump defect and swelling in the diabetic rat erythrocyte: effects on filterability and implications for microangiopathy. Proc Natl Acad Sci USA 1989; 86:3327-31.
15. Darlington D N, Gann D S. Adenosine Stimulates Na/K ATPase, and Prolongs Survival in Hemorrhagic Shock. J. Trauma. in press.
16. Abdel-Zaher A O, Abdel-Aal R A, Aly S A, et al. Adenosine for reversal of hemorrhagic shock in rabbits. Jpn J Pharmacol 1996; 72:247-54.
17. Griffith D A, Jarvis S M. Nucleoside and nucleobase transport systems of mammalian cells. Biochim Biophys Acta 1996; 1286:153-81.
18. Baldwin S A, Beal P R, Yao S Y, et al. The equilibrative nucleoside transporter family, SLC29. Pflugers Arch 2004; 447:73543.
19. Koc B, Erten V, Yilmaz M I, et al. The relationship between red blood cell Na/K-ATPase activities and diabetic complications in patients with type 2 diabetes mellitus. Endocrine 2003; 21:273-8.
20. Trunkey D D, Illner H, Wagner I Y, et al. The effect of septic shock on skeletal muscle action potentials in the primate. Surgery 1979; 85:63843.
21. Jones R O, Carlson D E, Gann D S. A circulating shock protein that depolarizes cells in vitro depresses myocardial contractility and rate in isolated rat hearts. J Trauma 1994; 37:752-8.
22. Blake P, Hasegawa Y, Khosla M C, et al. Isolation of “myocardial depressant factor(s)” from the ultrafiltrate of heart failure patients with acute renal failure. Asaio J 1996; 42:M911-5.
23. Shah K J, Chiu W C, Scalea T M, et al. Detrimental effects of rapid fluid resuscitation on hepatocellular function and survival after hemorrhagic shock. Shock 2002; 18:242-7.
24. Carrico C J, Coln C D, Lightfoot S A, et al. Extracellular Fluid Volume Replacement in Hemorrhagic Shock. Surg Forum 1963; 14:10-2.
25. Shires G T, Shires G T, III. Shock and Related Problems. Churchill Livingston, 1984:chap 2.
26. Gala G J, Lilly M P, Thomas S E, et al. Interaction of sodium and volume in fluid resuscitation after hemorrhage. J Trauma 1991; 31:545-55.
27. Blaustein M P. Sodium ions, calcium ions, blood pressure regulation, and hypertension: a reassessment and a hypothesis. Am J Physiol 1977; 232:C165-73.
28. Hamlyn J M, Blaustein M P, Bova S, et al. Identification and characterization of a ouabain-like compound from human plasma. Proc Natl Acad Sci USA 1991; 88:6259-63.
29. Ferrandi M, Manunta P, Balzan S, et al. Ouabain-like factor quantification in mammalian tissues and plasma: comparison of two independent assays. Hypertension 1997; 30:886-96.
30. Rybakowski J K, Lehmann W. Decreased activity of erythrocyte membrane ATPases in depression and schizophrenia. Neuropsychobiology 1994; 30:11-4.
31. Lang F, Busch G L, Ritter M, et al. Functional significance of cell volume regulatory mechanisms. Physiol Rev 1998;78:247-306.
32. Trump B F, Berezesky I K. Calcium-mediated cell injury and cell death. Faseb J 1995; 9:219-28.
33. Bruns, Nucleosides & Nucleotides, 10(5), 931-934 (1991).
34. Daly, J. Med. Chem., 25, 197 (1982).
35. Jacobson, et al., J. Med. Chem., 35, 407422 (1992).
36. Pantely, et al., Circulation, 82, 1854 (1990).
38. Comford E M and Oldendorf W H (1975) Independent blood-brain barrier transport systems for nucleic acid precursors. Biochim Biophys Acta 394: 211-219.
39. Mabley, et al. Am J Physiol Gastrointest Liver Physiol Vol 284, G138-144 (2003).
40. Liaudet, L., et al., Am J Respir Crit Care Med, Vol 164, 1213-1220 (2001).
41. Hasko, G., et al., J. Immunol, Vol. 164, 1013-1019 (2000).

### SUMMARY OF THE INVENTION

Some embodiments of the invention are directed to a method of preventing or treating hemorrhagic shock in an animalby administering to the animal a therapeutically effective amount of a purine nucleoside selected from the group including inosine, guanosine, deoxyinosine, and deoxyguanosine. In some embodiments the therapeutically effective amount of the purine nucleoside is from about 1 to about 100 mg per kg In some embodiments the purine nucleoside is administered intravenously, by infusion or injections.

Another embodiment of the invention is directed to a method of preventing or treating septic shock in an animal by administering to the animal a therapeutically effective amount of a purine nucleoside selected from the group including inosine, guanosine, deoxyinosine and deoxyguanosine. An embodiment is also directed to a method of raising Na/K ATPase activity in erythrocytes or other cells in an animal having lower than normal activity of Na/K ATPase activity, by administering an amount of a purine nucleoside selected from the group including inosine, guanosine, adenosine, deoxyadenosine, deoxyinosine, and deoxyguanosine sufficient to cause the Na/K ATPase activity to increase.

Another embodiment of the invention is a method of preventing or treating septic shock or hemorrhagic shock in an animal, by administering to the animal a therapeutically effective amount of erythrohydroxy3-nonyl-adenosine, or 6-thioinosine or 6-thioguanosine.

An embodiment is also directed to a method of contacting a cell having an Na/K ATPase with a purine selected from the group comprising adenosine, inosine, guanosine, deoxyadenosine, deoxyinosine and deoxyguanosine, such that the purine increases the Na/K ATPase activity.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 UV absorption spectrum of the active HPLC fraction number 5.

FIG. 2 Effects of the ultra-filtrate of the active fractions from rat dog and fetal calf serum (FCS) on Na/K ATPase activity. Each assay was run separately and included controls (buffer) and ouabain. Volumes were maintained equal in all tubes by adjusting with assay buffer. Values represent mean±SE, n=4 replicates for each group. Each assay was run at least three times; figures are representative of these assays.

FIG. 3 Inhibition by ouabain of Na/K ATPase stimulation by the rat plasma ultra-filtrate (FIG. 3a); commercial adenosine stimulation of Na/K ATPase (FIG. 3b) and ouabain-induced inhibition of Na/K ATPase stimulation by adenosine FIG. 3c). Each assay was run separately and included controls (buffer) and ouabain. Volumes were maintained equal in all tubes by adjusting with assay buffer. The values represent mean±SE, n=4 replicates for each group. Each assay was run at least three times; the figures are representative of these assays.

FIG. 4. The effect of 1 mM of various adenosine analogs on Na/K ATPase. Ouabain is 0.001 mM. Control is buffer. The values represent mean±SE of 4 replicates for each group. The assay was run 3 times and graph is representative of these three assays.

FIG. 5. The effect of adenosine receptor antagonists, Caffeine and Aminophylline on adenosine stimulation of Na/K ATPase. Values represent mean±SE of 4 replicates for each group. Assay was run 2 times and graph is representative of these two assays.

FIG. 6. The effect of the equilibrative nucleoside transport inhibitor, Dipyridamole (1 mM) on adenosine stimulation of Na/K ATPase. The values represent mean±SE of 4 replicates for each group. The assay was run 2 times and graph is representative of these two assays.

FIG. 7 The effect of a single dose of adenosine (0.5 mM in saline) administered in an infusion of a volume of fluid equal to that volume lost during hemorrhage on survival of rats in severe hemorrhagic shock as compared to saline alone (p<0.01).

FIG. 8 Inosine, guanosine, deoxyadenosine and deoxyguanosine stimulated Na/K ATPase in a dose-dependent manner, as did adenosine. The values represent mean±SE of 4 replicates for each group. Control is assay buffer. The assay was run three times, figure is representative of these assays.

FIG. 9 Effect of ouabain on stimulation of Na/K ATPase by purine nucleosides. The values represent mean±SE of 4 replicates for each group. The assay was run three times, figure is representative of these assays.

FIG. 10 Effect of the mono, di and triphosphate analogs of inosine, adenosine and guanosine, and their bases on Na/K ATPase. The control is assay buffer. Ouabain is 1 μM. The values represent mean±SE of 4 replicates for each group. The assay was run 2 times and graph is representative of these assays.

FIG. 11 The effect of adenosine receptor blockers caffeine or aminophylline on the stimulation of Na/K ATPase by inosine, adenosine, guanosine, deoxyadenosine or deoxyguanosine. INO-inosine, ADO-adenosine, GUA-guanosine, DA-deoxyadenosine, DG-deoxyguanosine. The values represent mean±SE of 4 replicates for each group. The assay was run 4 times and graph is representative of these assays.

FIG. 12 The effect of the equilibrative nucleoside transport inhibitor, dipyridamole (1 mM) on purine (1 mM) stimulation of Na/K ATPase. The values represent mean±SE of 4 replicates for each group. The assay was run 3 times and graph is representative of these assays.

FIG. 13 The effect of the equilibrative nucleoside transport inhibitor, NBTI 10 μM) on purine (1 mM) stimulation of Na/K ATPase. The values represent mean±SE of 4 replicates for each group. The assay was run 3 times and graph is representative of these assays.

FIG. 14 Kaplan-Meier Survival Analysis of rats resuscitated with either saline+2.5 mM inosine, adenosine, guanosine, cytidine or saline alone. 8 rats/group. Inosine, adenosine and guanosine groups survived significantly longer than saline or cytidine controls.

FIG. 15A) Changes in mean arterial blood pressure and B) heart rate before (−10 min) and immediately after resuscitation (from time 0 onward). The values represent mean±SE for each group. n=8 rats/group at time 0 but diminish with time as rats die.

FIG. 16 Kaplan-Meier Survival Analysis of rat in endotoxic shock (LPS, 10 mg/kg, iv) and resuscitated by continuous infusion (5 ml/hr) of either saline, inosine (5 mM), or nothing for 5 hrs. Inosine resuscitated rats survived significantly longer than the saline or no-resuscitation groups.

FIG. 17A) The effect of ouabain (10 mM), adenosine (1 mM), inosine (1 mM) and EHNA on Na/K ATPase activity in erythrocytes, and the effect of 6-thio inosine and 6-thio guanosine (both 1 mM) on Na/K ATPase activity.

### DETAILED DESCRIPTION

This invention relates to a method of treating or preventing hemorrhagic shock in an animal by administering intravenous inosine, guanosine, deoxyinosine, deoxyguanosine or a mixture thereof. It further relates to a method for treating an animal with severe blood loss by administering intravenous inosine, guanosine, deoxyinosine, deoxyguanosine or a mixture thereof to prevent hemorrhagic shock. A method is also described for treating or preventing septic shock in an animal by administering intravenous inosine, guanosine, deoxyinosine, deoxyguanosine or a mixture thereof. The invention is also directed to a method for increasing Na/K ATPase activity in erythrocytes or other cells in an animal having below normal activity of this enzyme by administering inosine, guanosine, deoxyinosine, deoxyguanosine or a mixture thereof.

In the following description, for the purposes of explanation, numerous specific details are set forth in order to provide a thorough understanding of the present invention. It will be apparent, however, to one skilled in the art that the present invention may be practiced without these specific details.

Severe hemorrhagic and septic shock are associated with a rise in plasma K (1,9,15,16), a rise in intracellular Na and water (9-13, 21), and a fall in intracellular K and membrane potential (6, 9-13, 21), all consistent with inhibition of Na/K ATPase. These changes could also result from limited availability of ATP, and initially were thought to be caused by decreased ATP. However, direct measurement of ATP revealed it to be increased, pointing to inhibition of the enzyme Na/K ATPase. The movement of Na and H₂O into cells and out of the extracellular space contributes to the fall in blood volume and blood pressure that is seen in hemorrhagic and septic shock. This probably explains why resuscitation with volumes larger than those lost during hemorrhage is necessary for cardiovascular stabilization (23-26). Although inhibition of Na/K ATPase has been recognized as a consistent feature of shock (2-13).

Na/K ATPase is a membrane bound protein found in all cells. It functions to maintain a chemical and electrical gradient across the cell membrane by moving Na out of and K into the cell. The energy that maintains the electro-chemical gradient comes from the hydrolysis of ATP to ADP. It is the energy in this gradient that drives other membrane transport, cotransport and exchange systems, allows the movement of cations, anions, amino acids and glucose across the cell membrane and maintains vital cellular functions including membrane electrical potential, cell volume and intracellular Na and Ca concentration. Na/K ATPase activity will vary in response to changes in the above mentioned variables in order to maintain low intracellular Na and the normal electrochemical gradient. Any substance or clinical condition that alters the normal activity of Na/K ATPase will change intracellular Na and Ca and the electro-chemical gradient, which will lead to a wide array of cellular dysfunctions that are attributed primarily to changes in cell volume (31, 32).

Purines such as adenosine have been shown to play a wide array of roles in biological systems. For example, physiological roles played by adenosine include, inter alia, modulator of vasodilation and hypotension, muscle relaxant, central depressant, inhibitor of platelet aggregation, regulator of energy supply/demand, responder to oxygen availability, neurotransmitter, and neuromodulator. (33). Because of its potent actions on many organs and systems, adenosine and its receptors have been the subject of considerable drug-development research (34). Potential therapeutic applications for agonists include the prevention of reperfusion injury after cardiac ischemia or stroke, and treatment of hypertension and epilepsy (35). Adenosine itself has recently been approved for the treatment of paroxysmal supra ventricular tachycardia (36). Others have found that adenosine was useful in treating hemorrhagic shock in rabbits. (37).

In order to assess further the role of purines and Na/K ATPase in hemorrhagic and septic shock, experiments were designed to look for compounds in fractionated plasma from shocked animals that affect Na/K ATPase. It was discovered that various fractions of plasma from normal animals (rat, pig and cow) subjected to hemorrhagic shock (hereafter “shocked animals”) and purified with liquid chromatography had the ability to stimulate red blood cell Na/K ATPase. The active substance was identified as adenosine.

The Na/K ATPase assay used is described in detail in Example 1A. The assay is done in red blood cells. Example 1B provides the method for obtaining an ultra-filtrate from plasma and serum. Example 1C describes the methods used to purify the ultra-filtrate using HPLC. The fraction containing adenosine was able to stimulate Na/K ATPase activity in human erythrocytes, assessed using 86RB uptake. Example 1D has a description of the rat hemorrhagic shock model that was used for in vivo testing to assess the ability of adenosine to prolong survival.

The results of the experiments described below showed that adenosine is present in HPLC purified ultra-filtrates of plasma from rat, pig and cow subjected to hemorrhagic shock. Adenosine stimulates Na/K ATPase activity in red blood cells, and this stimulation occurs even in the presence of ouabain, a specific inhibitor of Na/K ATPase (15).

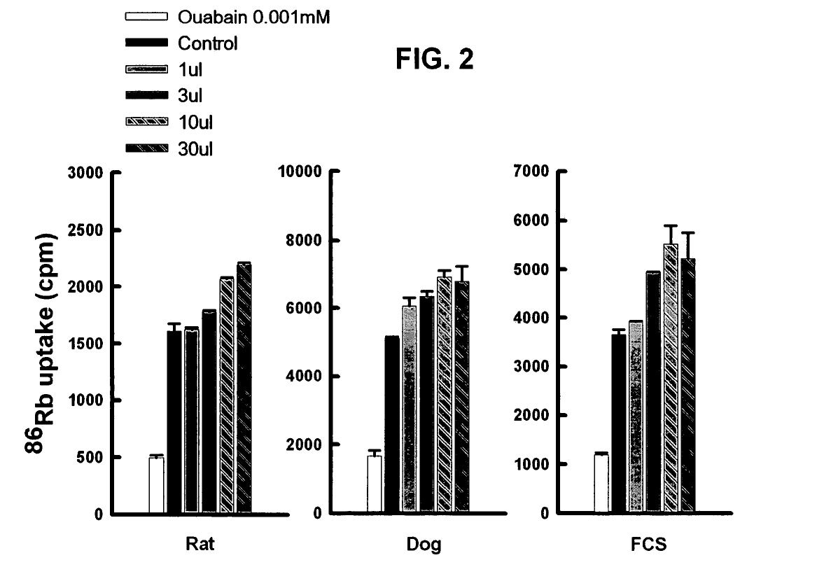

First, ultra-filtrates of rat and dog plasma, and of fetal calf serum (FCS) were collected and tested for the ability to stimulate Na/K ATPase activity in red blood cells. The ultra-filtrates all stimulated Na/K ATPase activity in a dose-dependent manner (FIG. 2). Each assay was run separately and included controls (buffer) and ouabain. Volumes were maintained equal in all tubes by adjusting with assay buffer. Values represent mean±SE, n=4 replicates for each group. Each assay was run at least three times; the figures are representative of these assays.

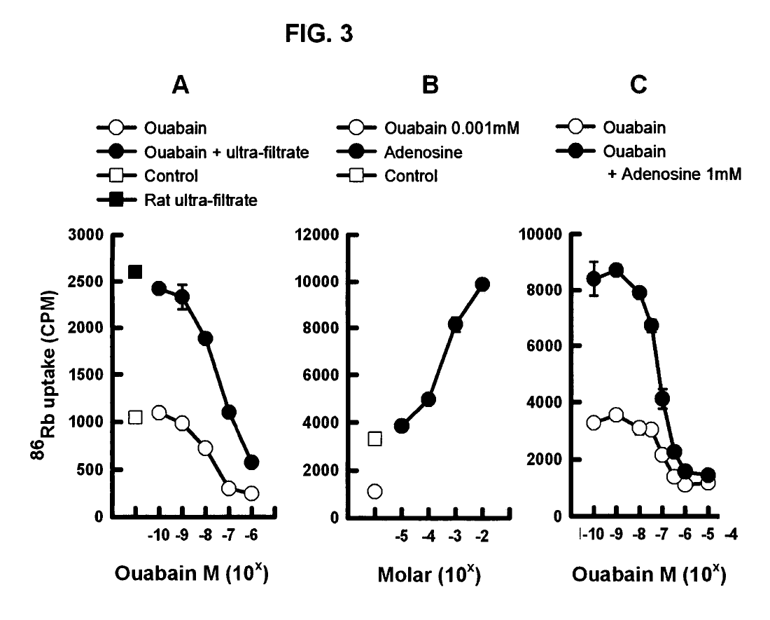

Stimulation of Na/K ATPase activity by the rat plasma ultra-filtrate was inhibited in a dose-dependent manner by ouabain (FIG. 3a), a specific inhibitor of Na/K ATPase. Each assay in FIG. 3 was run separately and included controls (buffer) and ouabain. Volumes were maintained at equal levels in all tubes by adjusting with assay buffer. Values represent mean±SE, n=4 replicates for each group. Each assay was run at least three times; the figures are representative of these assays.

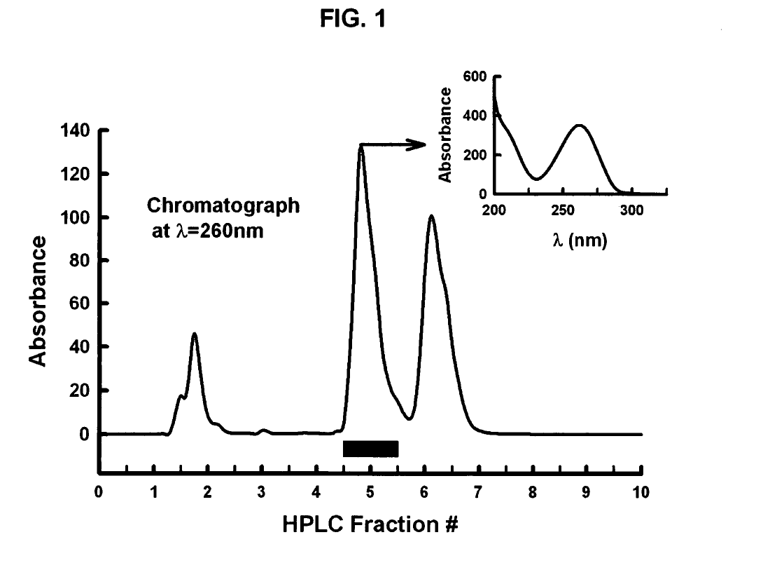

Using high performance liquid chromatography, the active fraction of the plasma ultra-filtrates (the fraction that stimulated Na/K ATPase) was purified using size exclusion, amine and hydrophilic-interaction chromatography. Adenosine was identified in the active fraction by its UV absorption spectrum. FIG. 1 Ultra-filtrates from fetal calf serum were separated by SEC HPLC and the active fractions sequentially separated on amine and hydrophilic interaction columns. FIG. 1 shows the chromatograph from the hydrophilic interaction column. The active fraction is marked by a bold line. The spectrum of that fraction is included in the inset.

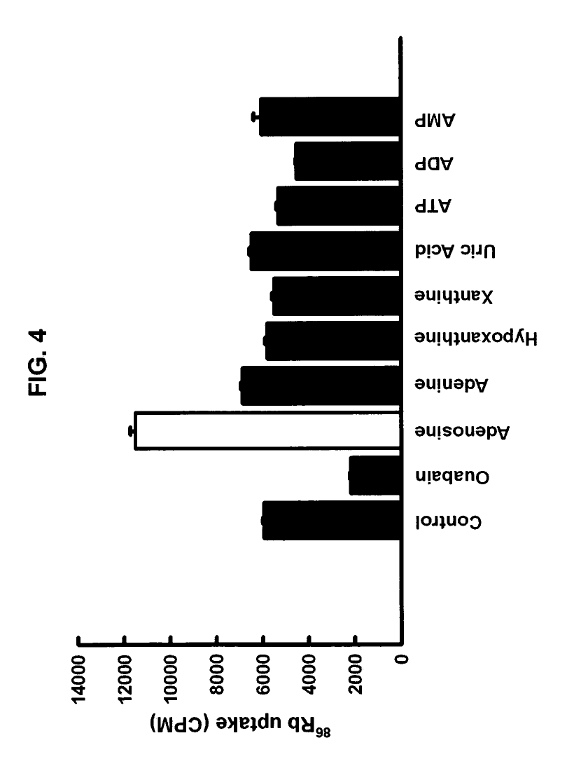

To determine if adenosine stimulated Na/K ATPase, commercial adenosine was substituted for the ultra-filtrate. Commercial adenosine stimulated Na/K ATPase in a dose-dependent manner (FIG. 3b) and the stimulation by adenosine was inhibited in a dose dependent manner by ouabain (FIG. 3c). This shows that adenosine stimulates Na/K ATPase. However, spectra of the active fragment described in FIG. 1 could also be an adenosine analog or metabolite (ATP, ADP, AMP, adenine, hypoxanthine, xanthine or uric acid) that has similar UV absorption spectra. To determine if adenosine is the principal substance that stimulates Na/K ATPase, each of these analogs were tested. The results of these experiments shown in (FIG. 4) demonstrate that the adenosine analogs and metabolites had little or no Na/K ATPase stimulatory activity. Thus, adenosine is the principal circulating substance that stimulates red blood cell Na/K ATPase.

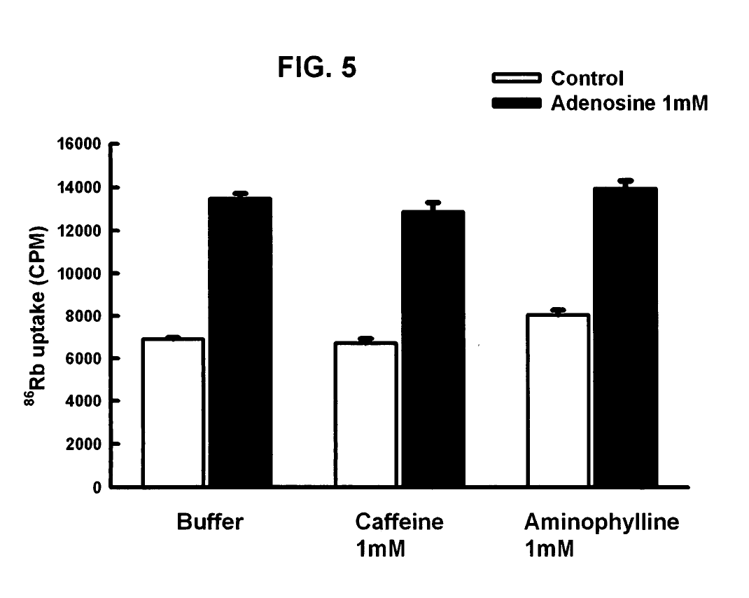

Many of the biological effects of adenosine are mediated through adenosine receptors. To determine if adenosine stimulates Na/K ATPase via adenosine receptors, equimolar doses of caffeine or aminophylline were added together with adenosine to the red blood cell assay. The adenosine-receptor blockers did not affect the ability of adenosine to stimulate Na/K ATPase (FIG. 5). Thus, the action of adenosine is independent of adenosine receptors.

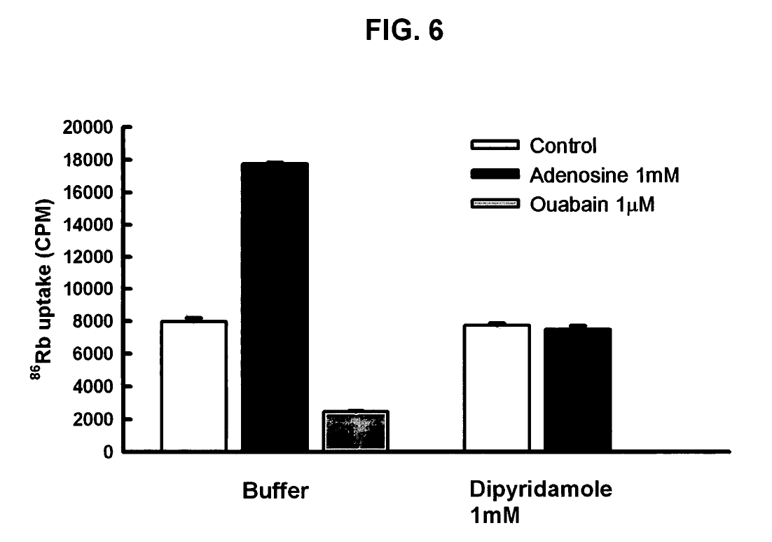

Adenosine is rapidly taken up by cells via an equilibrative nucleoside transporter in the cell membrane. The nucleoside transporter is the major mechanism by which adenosine is cleared from the plasma in vivo or from culture media in vitro. (17,18) Blockade of nucleoside transporters has been used to prevent disappearance of adenosine and extend the affect of adenosine stimulation. Dipyridamole was added together with adenosine to the red blood cell Na/K ATPase assay to determine if blocking the equilibrative nucleoside transporter potentiated the effects of adenosine by not allowing movement of adenosine into the cells. It was discovered that dipyridamole blocked adenosine stimulation of Na/K ATPase (FIG. 6). These data support the conclusion that adenosine action on Na/K ATPase is independent of adenosine receptors and that adenosine must enter the cell via the equilibrative nucleoside transporter to be effective.

Various hormones have been reported to stimulate Na/K ATPase activity in various cells types, including insulin, thyroxine, vasopressin, and catecholamines. These hormones were tested in the red blood cell Na/K ATPase assay. Insulin, thyroxine, vasopressin, epinephrine, norepinephrine and dopamine (in doses of 10⁻³ to 10⁻⁹M, varying orders of magnitude) had no effect on Na/K ATPase activity.

It was hypothesized that stimulation of Na/K ATPase with adenosine may reverse, alleviate or attenuate the effects of an endogenous inhibitor that appears during hemorrhagic shock. In order to test whether adenosine administration would prolong survival after hemorrhagic shock, adenosine was administered to rats in a single dose (0.5 mM in saline by intravenous infusion) as described in Example 1D. At such time that the rat would no longer maintain MABP at 35 mmHg and pressure began to fall (without further blood removal), infusion of fluid was given, containing either saline (150 mM NaCl) or adenosine (0.5 mM) in saline. The resuscitation volume was equal to the amount of blood removed (between 9-14 ml for both groups). MABP, heart rate and survival time were recorded. Survival of the saline verses adenosine groups was compared by Kaplan-Meier survival Analysis. Blood samples (0.5 ml) were taken before hemorrhage, before resuscitation and 1 hr after resuscitation for measurement of plasma K and Na.

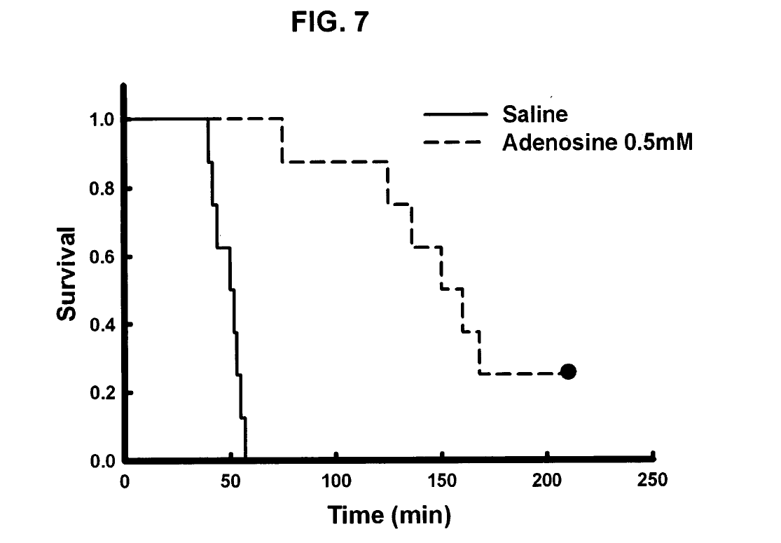

A single dose of adenosine (0.5 mM in saline) administered in an infusion of a volume of fluid equal to that volume lost during hemorrhage significantly prolonged survival of rats in severe hemorrhagic shock as compared to saline alone (p<0.01, FIG. 7). Resuscitation with adenosine resulted in an adenosine dose of 4.5 to 7 μMols (3 to 4.5 mg/k). No resuscitation resulted in death within 10 min (n=6). All of the saline resuscitated rats died within 60 minutes of resuscitation whereas the adenosine resuscitated rats survived significantly longer (FIG. 7). There was 100% survival at about 70 minutes, 85% from 70 to 120 minutes, and about 25% survival at about 160 minutes. Normally, prolonged survival is not seen in this hemorrhage model even with return of shed blood and additional electrolyte solutions (22-24). Resuscitation with adenosine could possibly extend the period of time before shock becomes irreversible, thus allowing for life saving surgical intervention.

All of the shocked rats showed a significantly elevated plasma K during shock and just prior to resuscitatior which is consistent with Na/K ATPase inhibition by shock. Resuscitation with adenosine led to the recovery of plasma K to prehemorrhage levels by 1 hr. Theses results agree with findings by Abdel-Zaher, et al. (16) who showed that adenosine resuscitation prolonged survival and corrected the elevation in plasma K cause by shock. The present findings support the hypothesis that inhibition of Na/K ATPase contributes to mortality of hemorrhagic shock.

Unfortunately, the single dose of 0.5 mM adenosine administered in the experiments just described caused bradycardia in the shocked rats. This necessitated a slow infusion to minimize the detrimental effects of a decreased cardiac output. The bradycardia was short lived and disappeared as soon as the infusion ceased or slowed. Irrespective of the detrimental effects on cardiac output, adenosine-treated rats survived significantly longer than saline resuscitated rats (FIG. 7). However, this adverse side effect, led us to test the effectiveness in stimulating the Na/K ATPase and in treating hemorrhagic shock of adenosine analogs.

Adenosine Analogs Stimulate Na/K ATPase and Resuscitate Animals After Hemorrhagic Shock Without any Adverse Effects

Because of the adverse side effect of adenosine causing bradycardia, the next series of experiments was conducted to test the ability of the purine nucleosides inosine, guanosine, deoxyadenosine, deoxyguanosine to stimulate Na/K ATPase. A dynamic equilibrium exists between adenosine which is metabolized to inosine which is converted to guanosine; and guanosine which is converted to inosine and then adenosine. The results of the experiments in this section showed that these inosine, guanosine, deoxyadenosine, and deoxyguanosine stimulated Na/K ATPase in a dose dependent manner, overcame partial inhibition by ouabain, and resuscitated rats after hemorrhagic shock. Furthermore, inosine and guanosine resuscitation from hemorrhagic shock did not cause the bradycardia that is seen with adenosine. It was further discovered that the de-ribosylated bases, (adenine, hypoxanthine and guanine), the nucleotides and the pyrimidines had little or no effect on Na/K ATPase activity.

To further summarize, inosine, guanosine, deoxyadenosine, and deoxyguanosine did not stimulate Na/K ATPase activity through adenosine receptors as caffeine or aminophylline did not block stimulation. Stimulation was blocked by inhibitors of the equilibrative nucleoside transporter (dipyridamole, 1 mM, or S-(4-nitrobenzyl)-6-thioinosine, NBTI, 10 μM), suggesting that the mechanism of action is intracellular. This blockade also excludes the A3 receptor, which is not blocked by caffeine or aminophylline, since there would be increased presentation of purine to the receptor by preventing cellular uptake. Inosine, guanosine and adenosine significantly increased survival of rats in hemorrhagic shock as compared to saline and cytidine controls, and lowered the shock-elevated plasma K⁺.

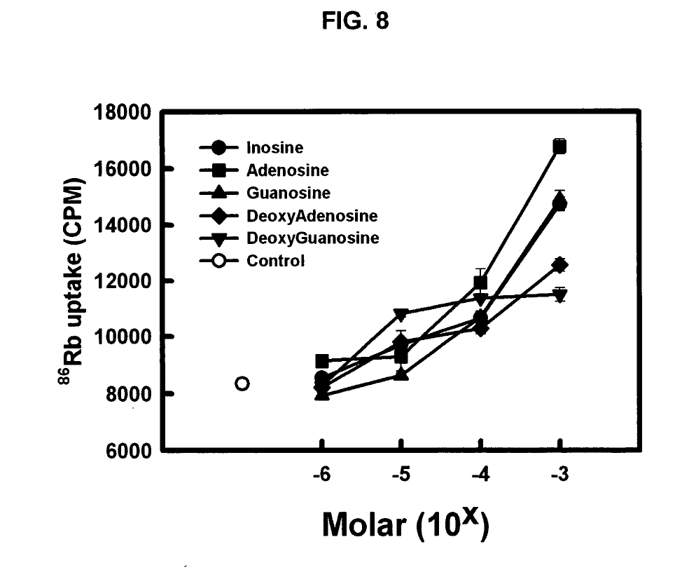

To determine if analogs of adenosine stimulate Na/K ATPase, natural purine nucleosides were added to the Na/K ATPase assay. Inosine, guanosine, and the analogs deoxyadenosine and deoxyguanosine stimulated Na/K ATPase in a dose-dependent manner, as did adenosine (FIG. 8). Stimulation by these compounds was very similar at doses between 10⁻⁴ and 10⁻⁶ Molar. At higher doses of 10⁻³ Molar, adenosine was more effective at stimulating Na/K ATPase than guanosine and inosine (which were both equally effective) Deoxyadenosine was less effective than guanosine and inosine, but more effective than deoxyguanosine. Values represent mean±SE of 4 replicates for each group. The control is assay buffer. The assay was run three times; the figure is representative of these assays.

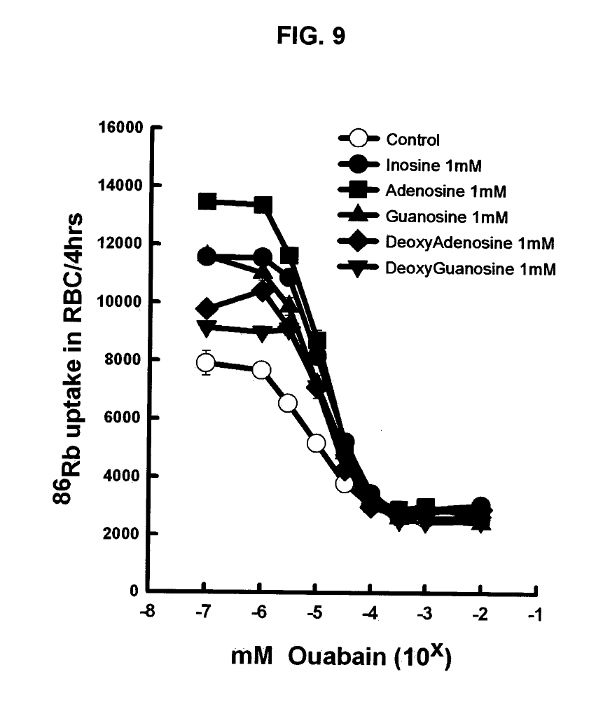

Stimulation of Na/K ATPase by inosine, guanosine, and the analogs deoxyadenosine and deoxyguanosine was inhibited by maximal doses of ouabain (FIG. 9), suggesting that the purines directly affect the activity of this enzyme. However, the purines were able to stimulate Na/K ATPase at sub maximal doses of ouabain (FIG. 9). Values represent mean±SE of 4 replicates for each group. The assay was run three times; the figure is representative of these assays.

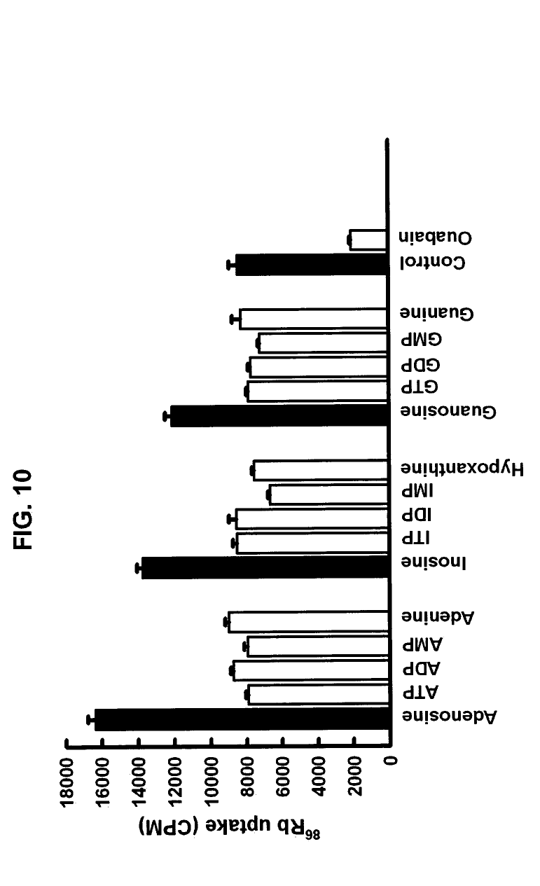

Other metabolic analogs of purine nucleosides were tested, including the mono, di and triphosphate analogs of inosine, adenosine and guanosine, and their bases (adenine, hypoxathine, guanine). None of the purine analogs significantly affected Na/K ATPase (FIG. 10). The control was assay buffer. The concentration of ouabain is 1 μM. Values represent mean±SE of 4 replicates for each group. The assay was run 2 times and the graph was representative of these assays. Moreover, the pyrimidines (cytidine, deoxycytidine, thymidine and uridine, in doses ranging from 10⁻³ to 10⁻¹⁰ M) had no effect in the assay. These data show that the purine heterocyclic structure is crucial for activity. These data also show that the absence of the ribose or the presence of phosphates eliminated the ability to stimulate Na/K ATPase in this assay.

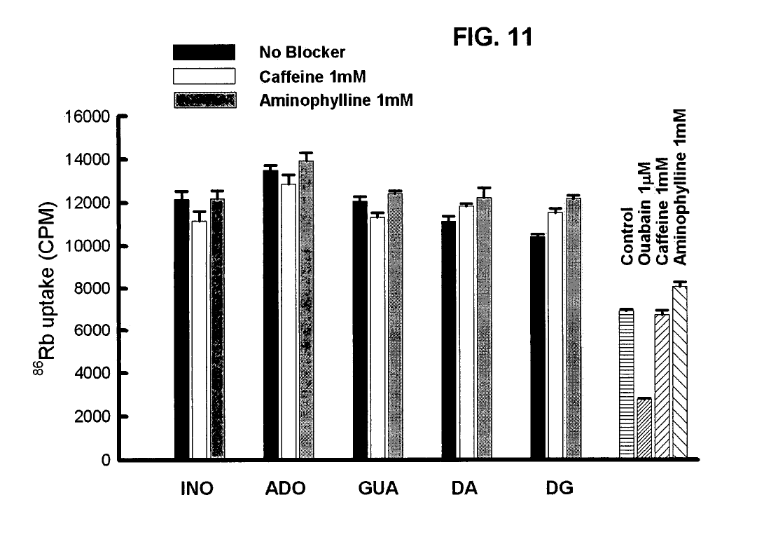

Some of the biological effects of purines are known to be mediated through adenosine receptors. To determine if adenosine receptors A1 and A2 are involved in the purine stimulation of Na/K ATPase, caffeine or aminophylline were added with one of inosine, adeno sine, guano sine, deoxyadenosine or deoxyguanosine. Neither of these adenosine-receptor blockers had a significant effect on stimulation of Na/K ATPase by any of the purines tested (FIG. 11). [INO-inosine, ADO-adenosine, GUA-guanosine, DA-deoxyadenosine, DG-deoxyguanosine.] Values represent mean±SE of 4 replicates for each group. The assay was run 4 times and the graph is representative of these assays. Thus these purines do not stimulate Na/K ATPase through adenosine receptors. A3 receptors are probably also not involved, for the reasons cited above.

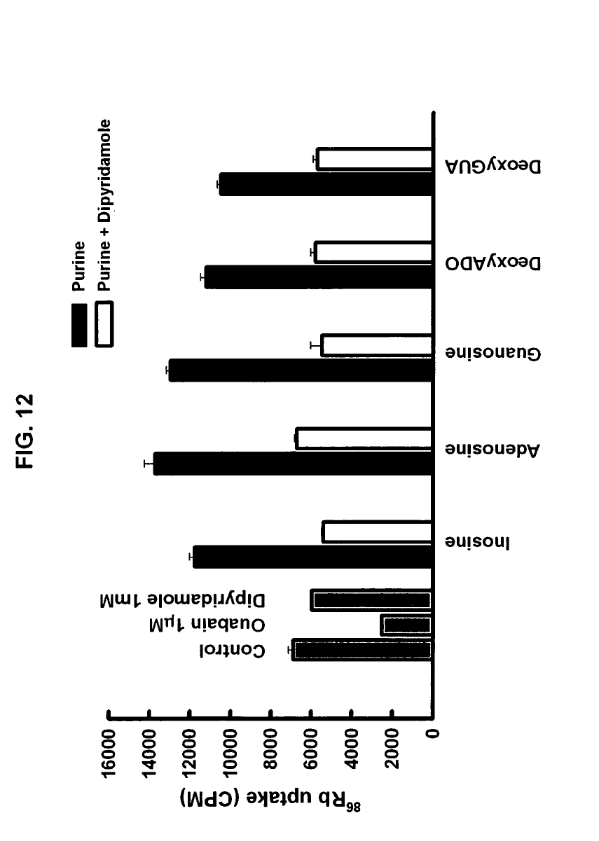

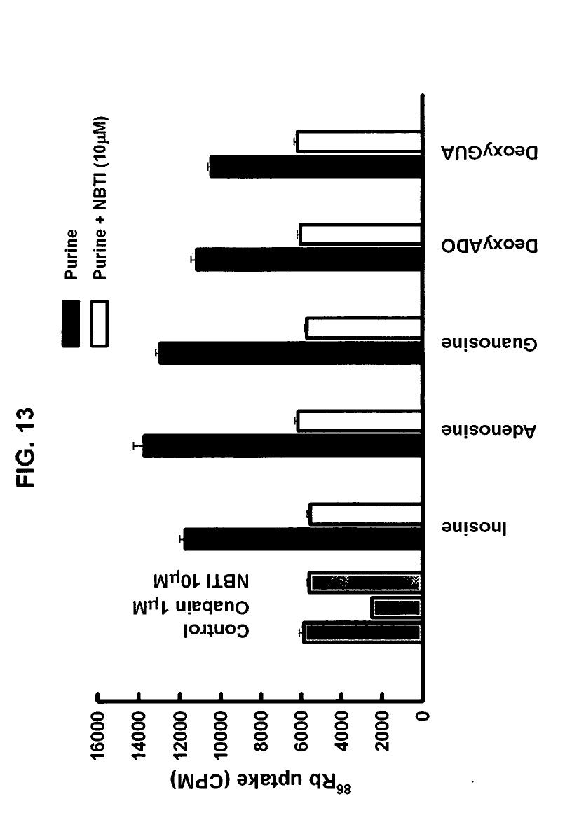

It was already shown that inhibitors of the nucleoside transporter prevent adenosine stimulation of Na/K ATPase. To determine if blockade of the equilibrative nucleoside transporter also affects stimulation of Na/K ATPase by inosine, guanosine, and the analogs deoxyadenosine and deoxyguanosine, either dipyridamole or NBTI were added together with the purines. The results showed that both dipyridamole (1 mM) and NBTI (10 μM) blocked stimulation of Na/K ATPase by any of the five purines tested (FIGS. 12 and 13). Values represent mean±SE of 4 replicates for each group. The assay was run 3 times and the graph is representative of these assays. These data show that purines stimulate Na/K ATPase intracellularly through a mechanism of action that is independent of adenosine receptors.

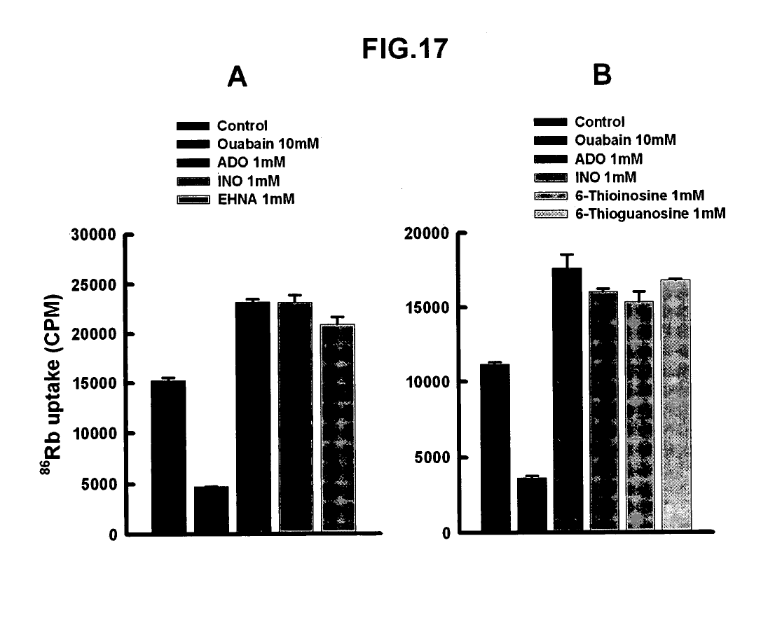

Other compounds that stimulate Na/K ATPase in the assay include EHNA (erythrohydroxy3-nonyl-adenine, an adenosine deaminase inhibitor (FIG. 17A), and 6-thioinosine and 6-thioguanosine (FIG. 17B). Any variant of the heterocyclic adenosine with ribose is a likely candidate for stimulating the Na/K ATPase, and may have therapeutic use.

Based on data showing that the purine heterocyclic structure and the ribose group are crucial for activity, embodiments of the present invention is directed to: the therapeutic use of inosine, guanosine, deoxyadenosine, deoxyinosine, deoxyguanosine, EHNA, 6-thioinosine and 6-thioguanosine, or mixtures thereof to treat diseases in an animal that are associated with lower than normal levels of Na/K ATPase activity in erythrocytes or other cells. An amount of these drugs is administered that increases Na/K ATPase activity, if possible to normal While adenosine caused bradycardia when administered at a rate of about 3 to 4.5 mg/kg by infusion, this side effect was eliminated by infusing at a slower rate. Therefore adenosine (administered at doses and rates that do not cause bradycardia) can also be used. Because the purines cross the blood brain barrier using a purine nucleoside-specific blood brain barrier transporter, they can be used in patients having diseases where brain Na/K ATPase is low. (39) Other analogs that have the purine heterocyclic structure and ribose and that stimulate Na/K ATPase can also be used. In a preferred embodiment, the above-identified compounds are administered at a dose of from about 1 to about 100 mg/kg/hr by any known route of administration including intramuscular, intravenous, intraperitoneal and oral or rectal administration. Unlike hemorrhagic or septic shock which are crisis modes, many diseases associated with lower than normal activity of Na/K ATPase are long term illnesses. A physician will be able to adjust the amount and rate of administration based on each patient's response.

The activity of Na/K ATPase in erythrocytes is decreased in patients with diabetes, and in animal models of diabetes. Generally speaking, in humans Na/K ATPase activity is down in Type 1 diabetes. Reports of a decrease in erythrocyte Na/K ATPase activity in Type II are mixed, with some laboratories showing a decrease in Na/K ATPase activity and some showing no change. Koc, B., Erten, V., Yilmaz, M. I., Sonmez, A., Kocar, I. H., The relationship between red blood cell Na/K-ATPase activities and diabetic complications in patients with type 2 diabetes mellitus, Endocrine 2003;21:273-8; Gamer, M H., Changes in Na,K-adenosine triphosphatase (ATPase) concentration and Na,K-ATPase-dependent adenosine triphosphate turnover in human erythrocytes in diabetes, Metabolism 1996; 45:927-34; De La Tour, D. D., Raccah, D., Jannot, M. F., Coste, T., Rougerie, C., Vague, P., Erythrocyte Na/K ATPase activity and diabetes: relationship with C-peptide level, Diabetologia 1998; 41:1080-4; Golfinan, L., Dixon, I. M., Takeda, N., Lukas, A., Dakshinamurti, K., Dhalla, N. S., Cardiac sarcolemmal Na(+)-Ca2+ exchange and Na(+)-K⁺ ATPase activities and gene expression in alloxan-induced diabetes in rats, Mol Cell Biochem 1998; 188:91-101; Leong, S. F., Leung, T. K. Diabetes induced by streptozotocin causes reduced Na-K ATPase in the brain, Neurochem Res 1991, 16:1161-5; Kowluru, R., Bitensky, M. W., Kowluru, A., Dembo, M., Keaton, P. A., Buican, T., Reversible sodium pump defect and swelling in the diabetic rat erythrocyte: effects on filterability and implications for microangiopathy. Proc Natl Acad Sci USA 1989; 86:3327-31.

Depression, bipolar disorder and schizophrenia are all associated with a decreased activity of Na/K ATPase in various cells including in erythrocytes, cardiac muscle and brain. See el-Mallakh et al., The Na,K-ATPase hypothesis for bipolar illness, Biol Psychiatry 1995; 37:235-44 (providing a review of evidence that supports a “cellular model for bipolar illness” based on the findings that Na/K ATPase activity is decreased in patients with bipolar disorder, mania and depression); see also Rybakowski J K, and Lehmann W, Decreased activity of erythrocyte membrane ATPases in depression and schizophrenia, Neuropsychobiology 1994; 30:11-4; el-Mallakh R. S., The Na,K-ATPase hypothesis for manic-depression. I. General considerations, Med Hypotheses, 1983; 12:253-68; el-Mallakh R. S., The Na,K-ATPase hypothesis for manic-depression. II. The mechanism of action of lithium. Med Hypotheses 1983; 12:269-82.

Patients with diabetes, depression, bipolar disorder and schizophrenia associated with low Na/K ATPase activity may benefit from treatment with purine nucleosides.

In Vivo Hemorrhagic Shock

Since purines can stimulate Na/K ATPase in the in vitro assay and overcome inhibition by ouabain, we designed in vivo experiments to test whether purines might stimulate Na/K ATPase in hemorrhagic shock, a pathophysiologic condition that generates endogenous inhibitors of Na/K ATPase.

Pentobarbital-anesthetized rats were subjected to severe hemorrhagic shock. Five groups of eight rats were resuscitated. The volume of resuscitation was equal to the volume of blood lost during the shock protocol (10.5±0.4, 11.0±0.5, 10.0±0.4, 10.3±0.6 and 10.9±0.4 ml, n=8/group, for saline, inosine, adenosine, guanosine, and cytidine, respectively). Hemorrhagic shock was induced by withdrawing blood from the femoral vein until mean arterial blood pressure (MABP) reached 35 mmHg (6-9 min) and withdrawn further to maintain MABP at 35 mmHg for 60 min. After 60 min, an infusion of a volume equal to that volume lost during hemorrhage and containing either saline (150 mM NaCl) or inosine, adenosine, guanosine, or cytidine (2.5 mM) in saline was infused over 8-9 minutes. This is about 16.75 mg/hr. This amount of NaCl delivered during resuscitation was the same between groups (ANOVA, 1.60±0.06, 1.65±0.08, 1.51±0.06, 1.55±0.09, 1.64±0.06 mmols for saline, inosine, adenosine, guanosine, and cytidine, respectively).

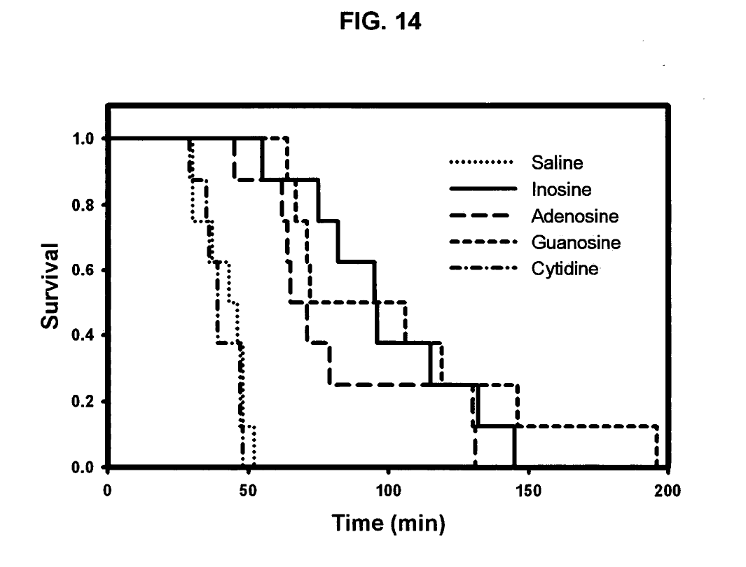

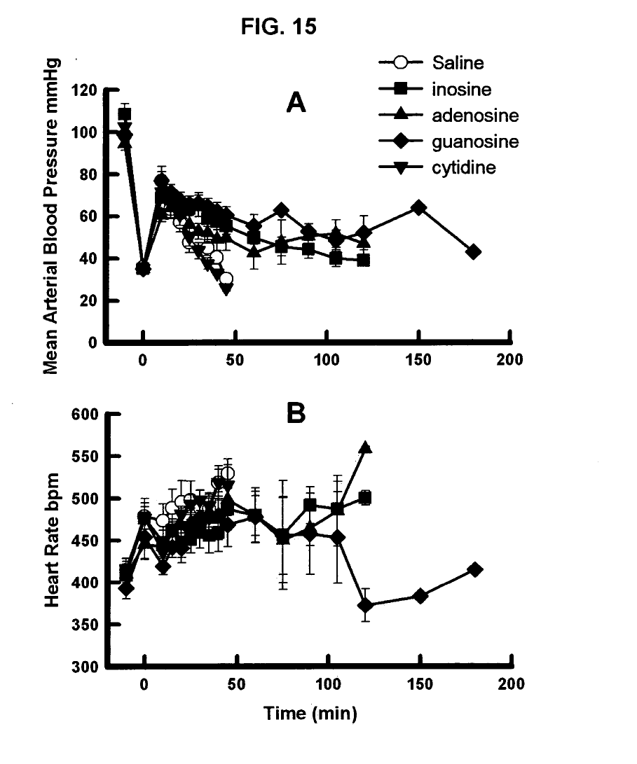

All of the saline and cytidine resuscitated rats died within 52 minutes of resuscitation, however rats resuscitated by administration of a single dose of 2.5 mM of inosine, adenosine or guanosine survived significantly longer than cytidine or saline controls (FIG. 14, p<0.001) as analyzed by Kaplan-Meier Survival Analysis. While 100% of shocked animals were dead at 50 minutes in saline or cytidine treated animals, animals treated with inosine did not suffer 50% mortality until about 100 minutes and 100% mortality was postponed until about 150 minutes. Animals treated with guanosine dropped to about 50% mortality sooner than with inosine but 100% mortality was postponed until 200 minutes. The changes in mean arterial blood pressure and heart rate were similar to the survival curves (FIG. 15). In all groups, there was an initial recovery of arterial blood pressure and heart rate after resuscitation. However, mean arterial blood pressure fell earlier in the saline and cytidine groups, mirroring the survival curves.

Plasma K⁺ was significantly elevated just prior to resuscitation as compared to plasma K⁺ measured before hemorrhage in all groups (Table 1). Resuscitation with purines lowered plasma K⁺ to prehemorrhage levels by 0.5 hr. By contrast, resuscitation with either saline or cytidine did not lower plasma K⁺ concentration. The appearance of hyperkalemia in severe shock could be interpreted as inhibition of Na/K ATPase by endogenous inhibitors that are elevated during hemorrhage. Purine administration reversed the hyperkalemia in vivo (Table 1).

TABLE 1
Plasma K⁺ (mM) before and at 0.5 hr after Resuscitation

| | Control | Shock | 0.5 hr after Resuscitation |
|---|---|---|---|
| Saline | 3.13 ± 0.26 | 4.40 ± 0.28* | 4.33 ± 0.34* |
| Inosine | 3.00 ± 0.18 | 4.10 ± 0.26* | 2.93 ± 0.21 |
| Adenosine | 3.17 ± 0.28 | 4.98 ± 0.67* | 3.10 ± 0.20 |
| Guanosine | 3.36 ± 0.18 | 4.31 ± 0.23* | 3.01 ± 0.15 |
| Cytidine | 3.03 ± 0.25 | 4.34 ± 0.38* | 4.30 ± 0.35* |

Plasma K⁺ was significantly (*) elevated (ANOVA, followed by Newman Kuels) after severe hemorrhagic shock. Resuscitation with purines lowered plasma K⁺ to levels not different from control.
N = 7/group.
Values are mean ± SE.
* represents significant difference from control (P < 0.05).

We have discovered that inosine or guanosine resuscitation of an animal in hemorrhagic shock lowers elevated plasma K⁺ and leads to significantly longer survival times after severe hemorrhagic shock than is observed in untreated animals and this occurs without any adverse effects. Therefore, one aspect of the present invention is the therapeutic use of inosine or guanosine to treat hemorrhagic shock. In a preferred embodiment, inosine or guanosine are administered intravenously at a dose of from about 1 to about 100 mg/kg/hr as a continuous infusion Alternatively, inosine or guanosine are administered by intravenous injections in an amount from about 1 to about 100 mg/kg as needed. Because inosine and guanosine are naturally occurring compounds, are metabolized to rapidly cleared metabolites and are rapidly cleared, high doses of up to 100 mg/kg/hr can be given if warranted by the symptoms. In the animal model, doses of about 16.75 mg/kg/hr resulted in complete recovery from hemorrhagic shock. The physician attending the patient will adjust the amount of the drugs administered over time depending on the patient's response. For very general guidance, guidance, an amount of about 500 mg/kg/day can safely be administered to a patient by intravenous injection or infusion. An “effective amount” is that dose which will ameliorate symptoms in the animal to which it is administered. When vital signs have returned to normal or to a level indicating the patient is out of danger, the drug treatment can be discontinued. Should a patient fail to recover with this dose, more can be administered since inosine and guanosine turn over rapidly to rapidly cleared metabolites.

Inosine has a short half life (<1 sec) in human blood. Purine nucleotides are rapidly taken up by all cells in the body via the equilibrative nucleoside transporter. Once inside cells, purine nucleosides are either 1) scavenged for production of ATP, or 2) degraded into hypoxanthine, xanthine and uric acid and excreted by normal mechanisms. Inosine and guanosine are metabolized and excreted by the body. As the patient recovers and shows more normal vital signs, the amount being administered can be decreased at the discretion of the doctor.

Where infusion is impractical, such as in crisis situations in auto accidents, on the battlefield, or where infusion equipment is not available, victims suffering from hemorrhagic shock or in danger of suffering from it, can be treated by multiple intravenous injections of inosine or guanosine as described above. Survival kits in the future may contain syringes of purine nucleosides to be administered in the event of hemorrhagic or septic shock. If the caregiver does not know how to administer an intravenous injection, the injection can be given intramuscularly or intraperitoneally. If administered intramuscularly, higher amounts and more frequent administrations are recommended. Injections can be given very frequently, even every five minutes, since inosine and guanosine are safe at relatively high doses. Oral administration is also possible.

Other embodiments of the present invention include prevention or treatment of hemorrhagic shock with deoxyadenosine, deoxyinosine, deoxyguanosine, 6-thioinosine, and 6-thioguanosine. FIG. 17B shows that 6-thioinosine and 6-thioguanosine also stimulate Na/K ATPase. Intravenous administration is preferred at a dose of from about 1 to about 100 mg/kg/hr, either as a continuous infusion or by one or more intravenous injections as needed. Administration can also be by intramuscular or intraperitoneal injection. Oral administration is also possible for prevention of shock. Routes of administration other than intravenous may be necessary in an emergency situation. In another embodiment, shock is treated with EHNA (erythrohydroxy3-nonyl-adenine, an inhibitor of adenosine deaminase) which also stimulates Na/K ATPase. Formulation of these compounds for therapeutic use is discussed below.

Septic Shock

Septic shock and cardiogenic shock are also associated with inhibition of Na/K ATPase. Eastridge B J, Darlington D N, Evans J A, Gann D S, A circulating shock protein depolarizes cells in hemorrhage and sepsis. Ann Surg 1994; 219:298-305; Trunkey D D, Illner H, Wagner I Y, Shires G T, The effect of septic shock on skeletal muscle action potentials in the primate. Surgery 1979; 85:638-43; Illner H P, Shires G T, Membrane defect and energy status of rabbit skeletal muscle cells in sepsis and septic shock. Arch Surg 1981; 116:1302-5; and Shires G T, 3rd, Peitzman A B, Illner H, Shires G T, Changes in red blood cell transmembrane potential, electrolytes, and energy content in septic shock. J Trauma 1983; 23:769-74.

One group has reported that inosine reduced inflammation and improved survival in a murine model of colitis. (40) Colitis was induced by dextran sulphate (DSS) sodium administration. Mice were given oral inosine either before the onset of colitis or as post treatment once colitis was established. Mice were given 200 gm/kg/day inosine orally twice per day on days beginning on days 1, or 4 or 7. By day 20 post colitis, 100% of the untreated rats died. Inosine protected the colon from DSS induced inflammatory cell infiltration and lipid peroxidation. The best results were obtained by administering inosine from day one, however, by day 30, 60% of the rats had nonetheless died. It should be emphasized that the DSS model of colitis is not a true septic shock model. It is a model for Crohn's disease or irritable bowel which is less life threatening.

Others have used inosine to treat septic shock induced by cecal ligation and puncture (CLP). Liaudet et al. (40) showed that inosine reduced systemic inflammation and improved survival. In these experiments mice were treated with three injections of inosine (100 mg/kg, intraperitoneally [IP]). There was a pre-treatment protocol with IP injections 1 hour before and 6 and 12 hours after CLP; and a post-treatment protocol with IP injections at 1, 12 and 24 hours after CLP for a total of 300 gm/kg over either 12 or 24 hours. Organ damage was significantly reduced by inosine treatment and survival was significantly improved. Untreated rats died at a rate of 60% b by the first 24 hours post-CLP, 80-85% at 48 hours, and essentially no survivors by 96 hours. Treated rats died at a slower rate. Rats pretreated with inosine died at a rate of 15% by 24 hours, and about 75% by 120 hours. In the post-treatment protocol there was about 20% mortality at 24 hours, but there was about 90% mortality at 120 hours.

In another study by Hasko, et al. inosine was shown to inhibit inflammatory cytokine production and it protected against lipopolysaccharide-induced shock (LPS, from E. Coli, serotype 055.B5) (41). Pretreatment with a single intraperitoneal injection of 100 mg/kg inosine 30 minutes before IP injection of a lethal dose of 70 mg/kg of LPS prolonged survival compared to untreated mice. Untreated mice died at a rate of 62.5% by about 10 hours, and about 94% by 36 hours. By contrast, 19% of mice pretreated with inosine died at 36 hours, 37.5% at 60 hours, and 50% by 96 hours. The eventual 50% mortality with IP administration of inosine is still unacceptable.

In an effort to obtain a more effective method of treating septic shock with purines, experiments were conducted administering inosine intravenously by infusion. Pentobarbital-anesthetized (50 mg/kg) rats (380-420 g) had cannulas placed in their femoral artery and vein. Arterial Blood pressure was monitored via the femoral artery cannula. After 15 minutes of stabilization, the rats were injected with 10 mg/kg of lipopolysaccharide (LPS, Sigma Aldrich, serotype 026:B6) in saline via the femoral vein. At the same time, an infusion of saline or inosine (5 mM) in saline or no infusion was begun (5 ml/hr) via the femoral vein cannula. A 5 mM solution of inosine is very concentrated. Arterial pressure and heart rate were monitored throughout the procedure. Survival time was recorded. Supplemental doses (15 mg/kg) of pentobarbital were given via the femoral vein cannula every hour. Rats surviving beyond 8.5 hr had their cannulas removed and were allowed to recover from pentobarbital anesthesia. These rats lived till 48 hours at which time we terminated the experiment. Inosine was administered at a rate of 1.34 mg/ml at 5 ml/hour for 5 hours which is a total of 33.5 mg for 5 hours. In 400 gram rats that is a total of about 83.75 mg/kg over the 5 hour period; 16.75 mg/hr or 402 mg/day.

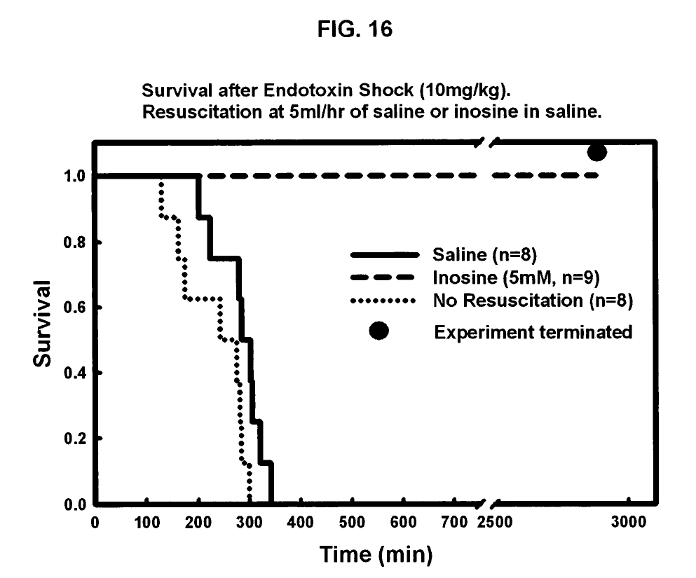

All inosine resuscitated rats lived and showed complete recovery with no signs of any difficulty whatsoever at 48 hours at which time they were sacrificed. FIG. 16. These results show that survival after intravenous infusion of inosine as described was 100% successful in resuscitating rats fully, with no sign of complications or distress. By contrast, a single IP injection of 100 mg/kg inosine before LPS-treatment reported in Hasko was much less effective (19% mortality at 36 hours and 37.5% mortality by 60 hours and 50% by 96 hours). In the Liaudet study rats subjected to CLP and treated with several IP injections of inosine died at a dramatically high rate of about 70% by 48 hours in the pre-treatment protocol and at an even higher rate of about 80% by 48 hours in the post-treatment protocol. Compared with these low survival rates obtained with IP injection of inosine, intravenous infusion of inosine is a highly superior method of treating septic shock.

Therefore, one aspect of the present invention is the therapeutic use of inosine to treat septic shock. In a preferred embodiment, inosine or guanosine or mixtures thereof are administered intravenously at a dose of from about 1 to about 100 mg/kg/hr as a continuous infusion. Alternatively, inosine or guanosine or mixtures thereof are administered by intravenous injections in an amount from about 1 to about 100 mg/kg as needed. Because inosine and guanosine are naturally occurring compounds, are metabolized to nontoxic metabolites and are rapidly cleared, high doses of up to 100 mg/kg/hr can be given if warranted by the symptoms. In the rat model, doses of about 16.75 mg/kg/hr for five hours resulted in complete recovery from shock. The physician attending the patient will adjust the amount of the drugs administered over time depending on the patient's response. For very general guidance, an amount of about 500 mg/kg/day can safely be administered to a patient. More can be given if a patient fails to respond since these nucleosides are naturally occurring and rapidly turned over into nontoxic metabolites. An “effective amount” is that dose which will ameliorate symptoms in the animal to which it is administered. However, septic shock will continue to be a problem until the bacteria that cause it have been eliminated, usually through antibiotic therapy. Therefore, treatment could go on for many days, theoretically even weeks. Again, the dose would be adjusted based on the patient's response.

Where infusion is impractical, such as in crisis situations in auto accidents, on the battlefield, or where infusion equipment is not available, victims suffering from shock or in danger of suffering from it, can be treated by multiple intravenous injections of inosine or guanosine in amounts of from about 1 to about 100 mg/kg. In the future, survival kits may include syringes of purine nucleosides to treat or prevent septic shock. If the caregiver does not know how to administer an intravenous injection, the injection can be given intramuscularly. If administered intramuscularly, higher amounts and more frequent administrations are recommended. Injections can be given very frequently, even every five minutes, until the patient is stabilized since inosine are safe at relatively high doses and turnover is quick.

In another embodiment, inosine and guanosine or other purine nucleosides are administered prophylactically to prevent septic shock in high risk patients such as those requiring gastrointestinal surgery or having wounds to the abdomen or intestines. In this embodiment inosine is administered intravenously by injections or infusions at a rate of between 1 to 100 mg/kg/hour before, during and/or after to prevent septic shock. Intestinal infection remains a major cause of morbidity and mortality throughout the third world and in travelers to the area. Translocation of bacteria from the intestine to the blood stream are serious complications of Salmonella typhi infections and translation of botulism toxin into the blood stream. IV administration of inosine or guanosine is a simple, inexpensive and highly effective method to treat or prevent deadly septic shock.

Inosine is highly soluble in water, therefore pharmaceutical formulations, discussed in more detail below, are straightforward for those skilled in the art to prepare. A survival kit could include syringes of inosine to be administered in the event of a serious infection and fever from a wound.

Other embodiments of the present invention include prevention or treatment of septic shock with deoxyadenosine, deoxyinosine, deoxyguanosine, 6-thioinosine, and 6-thioguanosine. Adenosine can be used if adjusted to lower doses that do not cause bradycardia. Intravenous administration is preferred at a dose of from about 1 to about 100 mg/kg/hr, either as a continuous infusion or by one or more intravenous injections as needed. Administration can also be by intramuscular or intraperitoneal injection. In another embodiment, septic shock is treated with EHNA (erythrohydroxy3-nonyl-adenine, an inhibitor of adenosine deaminase) which also stimulates Na/K ATPase.

### FORMULATIONS

Pharmaceutical formulations of the purines includes those suitable for intravenous ad ministration. The formulations may, where appropriate, be conveniently presented in discrete dosage units and may be prepared by any of the methods well known in the art of pharmacy. All such pharmacy methods include the steps of bringing into association the active compound with liquid carriers.

The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

The pharmaceutical composition preferably is administered by injection (intravenous or intramuscular), and the precise amount administered to a patient will be the responsibility of the attendant physician. However, the dose employed will depend upon a number of factors, including the age and sex of the patient, the precise disorder being treated, and its severity. Also the route of ad ministration may vary depending upon the condition and its severity.

Under conditions of severe blood loss, the composition and method are particularly beneficial in improving systemic and regional hemodynamics, and in improving tissue metabolism.

The method of the present invention may be performed by ad ministering the purines with or without a large volume of resuscitation fluid. The resuscitation fluid may suitably be Ringers-Lactate, a sodium chloride solution, red blood cells, whole blood, plasma, a crystalloid solution, a colloid solution, dextrose, albumin, ethanol, dimethylsulfoxide, water or combinations thereof.

For the purposes of this invention, the compounds of the invention may be administered by a variety of means including in formulations containing conventional non-toxic pharmaceutically acceptable carriers, adjuvants and vehicles. The term parenteral as used herein includes sub-cutaneous, intravenous, intramuscular, and intra-arterial injections with a variety of infusion techniques. Intra-arterial and intravenous injection as used herein includes administration through catheters. Preferred for certain indications are methods of administration which allow rapid access to the tissue or organ being treated, such as intravenous injections. When an organ outside a body is being treated, perfusion is preferred.

Pharmaceutical compositions containing the active ingredient may be in any form suitable for the intended method of administration.

The pharmaceutical compositions of the invention may be in the form of a sterile injectable preparation, such as a sterile injectable aqueous or oleaginous suspension. This suspension may be formulated according to the known art. A person of skill in the art will also know how to make formulations for oral, rectal, topical or other routes of administration.

The amount of active ingredient that may be combined with the carrier material to produce a single dosage form will vary depending upon the host treated, the particular mode of administration, and the active ingredient used. It will be understood that the specific dose for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

### EXAMPLES

### Example 1A

### Na/K ATPase Assay

Human red blood cells of any group-type were donated for research by the Blood Bank of the University Of Maryland, School of Medicine. Five ml of red cells are washed 3 times in 40 ml of Potassium free Ringers solution (141 mM NaCl, 1 mM H₂NaPO₄, 0.5 mM CaCl₂, 1 mM MgSO₄, 4.5 mM glucose, 10 mM HEPES pH7.4). 50 μl of washed red cells are added to 220 μl of potassium free Ringers solution. 30, 10, 3 or 1 μl of ultra-filtrate, HPLC (high performance liquid chromatography) fraction, or adenosine, inosine, guanosine, deoxyadenosine, deoxyguanosine, caffeine, aminophylline, dipyridamole, NBTI, 6thioinosine, 6thioguanosine, or EHNA (all in buffer) is added to the mixture and incubated for 20 min. 4 μCi of 86RbCl is added and incubated for 4 hrs at 37° C. The mixture is than washed three times with 4° C. Potassium free Ringers solution and the cells counted in a gamma counter (Packard, Cobra II). Na/K ATPase activity is expressed as the CPM (counts per minute) (CPM/tube*4 hrs). The effects of ultra-filtrate or HPLC fractions on ⁸⁶Rb uptake into red cells are compared to equal volumes of either water (control) or, in some cases, 0.01 mM Ouabain (maximal inhibition). All chemicals were purchased from Sigma Aldrich (St. Louis Mo.).

### Example 1B

### Ultra-Filtrate from Plasma and Serum

Whole blood is collected from cannulated femoral arteries of pentobarbital anesthetized dogs, or from unanesthetized rats that have indwelling cannulas in the femoral artery. The whole blood is centrifuged at 3000 rpm at 4° C. for 20 minutes to remove the cellular component. Alternatively, fetal calf serum is used. The plasma or serum is placed in Centriprep™ 30 (Millipore Corp. MA) and centrifuged at 200 rpm at 4° C. 2 hrs to remove proteins over 30K MW (molecular weight). This filtrate is then placed over Sep-Pac C18™ cartridges (Waters Corp, MA), and the flow-through collected and called ultra-filtrate.

### Example 1C

### Purification of Ultra-Filtrate by HPLC

Further fractionation was performed using sequential chromatography that included size exclusion (SHODEX™, Phenomenex, CA), amine (Phenomenex, CA) and hydrophilic interaction (PolyLC, MD) chromatography on HPLC, assaying each fraction for Na/K ATPase activity. The active fraction was isolated and identified by UV absorption as adenosine or one of its analogs (FIG. 1). Adenosine purchased from SigmaAldrich was shown to stimulate Na/K ATPase in a dose-dependent manner and to have the same retention time as the active fraction on all three HPLC columns.

### Example 1D

### Rat Shock Model for Adenosine Experiments

All animal procedures are conducted with the authorization of the Institutional Animal Care and Use Committee at the University of Maryland, School of Medicine. Male Sprague-Dawley Rats (385-450 g) are anesthetized with Pentobarbital-Sodium (50 gm/kg) and cannulas are placed in the femoral artery (PE-50) for measurement of arterial blood pressure, and vein (PE-90) to withdrawal blood and infuse resuscitation fluid. After 15 minutes of stabilization, blood is drawn from the femoral vein until mean arterial blood pressure (MABP) reached 35 mmHg and withdrawn further to maintain MABP at 35 mmHg. At such time that the rat would no longer maintain MABP at 35 mmHg and pressure began to fall (without further blood removal), an infusion of a volume equal to that volume lost during hemorrhage and containing either saline (150 mM NaCl) or adenosine (0.5 mM) in saline was started by gravity (column 4 inches above rat). MABP, heart rate and survival time are recorded. Survival of the saline verses adenosine groups was compared by Kaplan-Meier survival Analysis. Blood samples (0.5 ml) were taken before hemorrhage, before resuscitation and 1 hr after resuscitation for measurement of plasma K and Na. Blood was centrifuged 3500 rpm for 2 min, plasma removed and K and Na was measured by flame photometer. Before and after resuscitation potassium (K) was analyzed by Newman Keul post hoc test after ANOVA.

Rat Shock Model for Experiments with Adenosine Analogs

All animal procedures were conducted as described for adenosine experiments except that after 15 minutes of stabilization, blood was withdrawn from the femoral vein until mean arterial blood pressure (MABP) reached 35 mmHg (6-9 min) and withdrawn further to maintain MABP at 35 mmHg for 60 min. After 60 min, an infusion of a volume equal to that volume lost during hemorrhage (8.5-13 ml) and containing either saline (150 mM NaCl) or inosine, adenosine, guanosine, or cytidine (2.5 mM) in saline was infused over 8-9 min. MABP, heart rate and survival time were recorded. Plasma K⁺ was measured from plasma taken before hemorrhage, before resuscitation and 0.5 hrs after resuscitation using a K⁺ electrode (Jenco Electronics, Inc., San Diego, Calif.). Survival time between groups was compared by Kaplan-Meier survival Analysis.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

1. A method of preventing or treating hemorrhagic shock in an animal, comprising administering to the animal a therapeutically effective amount of a purine nucleoside selected from the group comprising inosine, guanosine, deoxyinosine, and deoxyguanosine.

2. The method of claim 1, wherein the purine nucleoside is administered intravenously.

3. The method of claim 1, wherein the purine nucleoside is administered by intravenous by infusion.

4. The method of claim 1, wherein the purine nucleoside is administered at a rate of from about 1 to about 100 mg per kg per hour.

5. The method of claim 1, wherein the purine nucleoside is administered as one or more intravenous injections, and the therapeutically effective amount is from about 1 mg to about 100 mg per kilogram per injection.

6. The method of claim 1 for preventing hemorrhagic shock in an animal, wherein the therapeutically effective amount of the purine nucleoside is from about 1 to about 100 mg per kg per hour and administration begins before the animal has developed hemorrhagic shock.

7. A method of preventing or treating septic shock in an animal, comprising administering to the animal a therapeutically effective amount of a purine nucleoside selected from the group comprising inosine, guanosine, deoxyinosine, and deoxyguanosine.

8. The method as in claim 7, wherein the purine nucleoside is administered intravenously.

9. The method of claim 7, wherein the purine nucleoside is administered by intravenous by infusion.

10. The method of claim 7, wherein the purine nucleoside is administered at a rate of from about 1 to about 100 mg per kg per hour.

11. The method of claim 7, wherein the purine nucleoside is administered as one or more intravenous injections, and the therapeutically effective amount is from about 1 mg to about 100 mg per kilogram per injection.

12. The method of claim 7, wherein the animal has a bacterial infection.

13. The method of claim 7 for preventing septic shock, wherein the animal has undergone gastrointestinal surgery and the therapeutically effective amount of the purine is administered to the animal after gastrointestinal surgery.

14. The method of claim 7 for preventing septic shock, wherein the animal is about to undergo gastrointestinal surgery and the therapeutically effective amount of the purine is administered to the animal before gastrointestinal surgery.

15. A method of raising Na/K ATPase activity in erythrocytes or other cells in an animal having lower than normal activity of Na/K ATPase, comprising administering to the animal an amount of a purine nucleoside selected from the group comprising inosine, guanosine, deoxyinosine, and deoxyguanosine sufficient to cause the Na/K ATPase activity to increase.

16. The method as in claim 15, wherein the purine nucleoside is administered at a rate of from about 1 to about 100 mg per kg per hour.

17. The method as in claim 15, wherein the purine nucleoside is administered intravenously.

18. A method of preventing or treating septic shock or hemorrhagic shock in an animal, comprising administering to the animal a therapeutically effective amount of erythrohydroxy3-nonyl-adenine.

19. A method comprising contacting a cell having an Na/K ATPase with a purine nucleoside selected from the group comprising adenosine, inosine, guanosine, deoxyadenosine, deoxyinosine and deoxyguanosine, wherein the purine causes an increase in activity of the Na/K ATPase.

20. A method of preventing or treating septic shock or hemorrhagic shock in an animal, comprising administering to the animal a therapeutically effective amount of 6-thioinosine and 6-thioguanosine.

21. A method for preventing or treating septic shock in an animal, comprising administering a therapeutically effective amount of a purine nucleoside selected from the group comprising adenosine and deoxyadenosine.
